(19) United States
(12) Patent Application Publication
(10) Pub. No.: US 2003/0109011 A1
(43) Pub. Date: Jun. 12, 2003

(54) Methods for the cost-effective saccharification of lignocellulosic biomass

(76) Inventors: Elizabeth E. Hood, College Station, TX (US); John A. Howard, College Station, TX (US)

Correspondence Address: Patricia A. Sweeney, 1835 Pleasant St., West Des Moines, IA, 50265-2334, US

(21) Appl. No.: 10/310,292

(22) Filed: Dec. 6, 2002

Related U.S. Application Data
(60) Provisional application No. 60/340,035, filed on Dec. 6, 2001.

(51) Int. Cl. C12P 19/02; A01H 1/00

(52) U.S. Cl. 435/105; 800/284

(57) ABSTRACT

The invention provides methods for the cost-effective saccharification of polysaccharides in lignocellulosic biomass, particularly in crop residues. In one embodiment of the invention polysaccharide-degrading enzymes are expressed in the seeds of plants, preferably in germ (embryo) tissue of seed. Corn crop plants are used in one embodiment of the invention. The corn seeds and the corn stover are harvested concurrently in a single pass harvesting operation to lower costs. The corn seeds are fractioned which allows for additional uses for separated tissue. The endosperm can be used as a source of starch for existing industries to produce by-product credits. In an embodiment, the starch is used to produce ethanol in currently existing facilities and the tissue, preferably germ, containing the polysaccharide-degrading enzymes can be used as the enzyme source. The appropriate tissues that express polysaccharide-degrading enzymes, or extracts thereof, are combined with the corn stover and the combination is exposed to conditions favorable for the conversion of the cell wall polysaccharides in the corn stover into fermentable sugars. The fermentable sugars can then be utilized by microorganisms to produce ethanol or other desired fermentative products.

### CROSS-REFERENCE TO RELATED APPLICATIONS

This application claims the benefit of U.S. Provisional Application No. 60/340,035, filed Dec. 6, 2001, which is hereby incorporated herein in its entirety by reference.

### FIELD OF THE INVENTION

The invention relates to the field of biotechnology, particularly to cost-effective methods for the saccharification of lignocellulosic biomass. The invention further relates to the genetic engineering of plants for use in such saccharification methods. In addition, the invention relates to cost-effective measures for producing ethanol from crop residues. The invention also relates to the commercial production of heterologous proteins in plants. More specifically the invention relates to methods of overexpressing heterologous polysaccharide-degrading enzymes in corn plants.

### BACKGROUND OF THE INVENTION

Fossilized hydrocarbon-based energy sources, such as coal, petroleum and natural gas, provide a limited, non-renewable resource pool. Because of the world's exponentially increasing population and increasing dependence on energy, this resource on which our standard of living depends, will likely be severely limited within the next 50 to 100 years. World crude oil reserves equal 981.4 billion barrels, and world usage is approximately 75 million barrels per day, (“Annual Energy Outlook 2001 With Projections to 2020”, Energy Information Admin., Dept. of Energy, Report No. DOE/EIA-0383) The U.S. transportation sector alone uses 100 billion gallons of gasoline per year. Most of the oil used in the U.S. today is imported, creating a somewhat dangerous situation in today's political climate, because disruptions are highly likely and any disruption in that supply would affect the ability of the economy to function.

Renewable-energy sources, such as those derived from plants, may provide a viable alternative to fossil fuels. For example, ethanol can be produced from plants via fermentation. In fact, the U.S. currently manufactures approximately 1.8 billion gallons of ethanol from corn grain-derived starch (Sheehan, J. (2001) “The road to bioethanol: A strategic perspective of the U.S. Department of Energy's National Ethanol Program,” In: Glycosyl Hydrolases for Biomass Conversion, Himmel, Baker, and Saddler, eds., American Chemical Society, Washington, D.C., pp. 2-25). Ethanol that is produced from corn starch, however, is not a cost-effective alternative to fossil fuels. Currently, the U.S. government subsidizes ethanol production from corn starch because the market price for ethanol is below the manufacturing cost.

Because known technologies for ethanol production from plant biomass are more costly than the market price for ethanol, ethanol will not become an important alternative to fossil fuels, unless the price of fossil fuels rises substantially. If, however, the cost of the production of ethanol from plant biomass could be reduced, then ethanol might become a cost-effective alternative to fossil fuels even at today's prices for fossil fuels.

As discussed above, the predominant method for ethanol production in the U.S. involves the use of starch that is derived from corn grain. The present U.S. production levels of corn grain are not sufficient to meet the projected demand for ethanol. If all corn grain produced in the United States could be used for ethanol, the yield would still not meet the projected demand for this fuel (10 billion gallons for 2001 usage rate) even if it is only used as a 10% blend with gasoline (Sheehan, J. (2001) supra). Therefore, other sources of plant biomass for fermentation into ethanol would likely be needed to meet the projected demand for ethanol.

While corn grain is currently the predominant form of plant biomass that is utilized for ethanol production, the most abundant source of plant-based renewable resources lies in the recovery of five and six carbon sugars from plant cell walls. Thus, any strategy designed to substitute biomass feedstocks for petroleum in the manufacture of fuels and chemicals must be able to efficiently convert the polysaccharide components of plant cell walls to soluble, monomeric sugar streams. Biomass feedstocks that are primarily comprised of plant cell walls are known as lignocellulosic feedstocks. Some under-utilized materials that can potentially be used as lignocellulosic feedstocks include, for example, crop residues such as plant stalks, husks, hulls, and leaves, residual forest biomass such as stumps, branches, foliage, and bark, mill wastes such as saw dust, edgings, end cuts and other wood scraps, and wood, particularly from trees that are not valuable for timber and pulp.

More than 400 million acres of land are employed for crop production in the United States and an additional 650 million acres are occupied by forests, some of which are cultivated for commercial uses (Walsh (1999) Biomass Feedstock Availability in the United States. State Level Analysis, Oak Ridge National Laboratory, Oak Ridge, Tenn., Apr. 30, 1999, updated January, 2000). Unharvested residues from agricultural crops are estimated at a mass approximately equal to the harvested portion of the crops. Thus, these unused crop residues are an abundant source of biomass that could be utilized for the production of fermentable sugars that can serve as precursors for the synthesis of a variety of chemical compounds such as ethanol. For example, if half of the crop residues from the U.S. corn crop were to be used for the production of renewable fuels, then about 120 million tons of corn stover would be available as a feedstock for biomass conversion processes (Walsh (1999) supra). Assuming that corn stover is approximately 40% cellulose on a dry weight basis, then 48 million tons of cellulose/year would be available for hydrolysis and conversion to glucose. A ton of cellulose will yield 122 gallons of ethanol if one makes the following assumptions: (1) cellulose hydrolysis is 80% efficient; (2) glucose fermentation to ethanol is 90% efficient; (3) the yield of ethanol from glucose is 51% (theoretical maximum) on a mass basis; and (4) the density of ethanol is 0.7893 kg/L. Therefore, 120 million tons of corn stover will yield 14 billion gallons of ethanol. Considering the other cellulose-containing materials other than corn stover, available for conversion, the goal of deriving 10% of the liquid fuels from renewable resources by the year 2020 (see Plant/Crop-based Renewable Resources Vision 2020: A Vision to Enhance U.S. Economic Security Through Renewable Plant/Crop-Based resouce Use (http://www.oit.doe.gov/agriculture/) appears to be achievable. However, this goal will only be achieved if ethanol production from corn stover and other lignocellulosic materials becomes cost-effective.

The economics of using corn stover or any other source of lignocellulosic biomass to produce ethanol is ominous at best and is the limiting step behind the attainment of such a goal. The current cost of making ethanol from any source of lignocellulosic biomass with the current enzyme production systems and the biomass collection and pretreatment technology is in the order of about $1.50 per gallon. This is due to the high operation costs of collecting and transporting the lignocellulosic raw material to destination plants, producing the polysaccharide-degrading enzymes and the high cost of pretreating the lignocellulosic raw material to facilitate its enzymatic degradation. To become economical, the processes for ethanol production have to be integrated into the cultivation of agricultural crops. In particular, the process of producing the enzymes required for ethanol production as well as the collection of lignocellulosic raw material have to be integrated into the normal operations of crop cultivation. The crop market will generate the revenues necessary to economically justify its cultivation and the production of ethanol will be a by-product of this operation.

Furthermore, the implementation of harvest methods that allow the simultaneous recovery of corn stover and corn grain by a single pass through the field reduces the cost of collecting the lignocellulosic raw material. Such single pass (also referred to as one-pass) harvesting cuts down on the number of times that farm machinery are driven through the fields. This approach minimizes soil compaction, reduces the amount of time invested in material collection and curtails the cost of fossil fuel and labor needed for operating the farm machinery. One-pass harvest is being developed by several groups, for example at Iowa State University by Dr. Graeme Quick. See records and minutes of the “Corn Stover Harvesting Field Demonstration and Biomass Harvesting Colloquium”, Harlan, Iowa. Oct. 29, 2001.

### BRIEF SUMMARY OF THE INVENTION

Provided by the invention are cost-effective methods for the saccharification of polysaccharides in crop residues. The methods of the invention find particular use in the integration of current practices for the cultivation of crop plants for the purpose of obtaining a commercially desired plant material with the production of commercial levels of polysaccharide-degrading enzymes in the tissues of the crop plants and the use of the crop plant residues as a source of lignocellulosic biomass for the production of fermentable sugars.

The methods of the invention find use in transforming crop plants with a nucleotide sequence encoding at least one polysaccharide-degrading enzyme. Preferrably the crop plant is a plant that produces seeds. The source of the enzyme preferably can be seed tissue, such as one or more of whole seed, hulls, seed coat, endosperm, or embryo (germ). More preferrably the seeds have a germ that is capable of being fractioned from the rest of the seed (the term degerminated is sometimes used when referring to separation of the germ) in a commercial milling process. In a preferred embodiment of the invention the enzyme(s) are expressed in the germ portion of the seed. In another preferred embodiment the level of enzymes that are produced in the germ portion of the the seed are at least about 0.1% of the dry weight of the seed.

In particular, the methods of the invention further provide a cost-effective integrated approach to producing fermentable sugars from corn stover that encompasses the production of polysaccharide degrading enzymes in the seeds of genetically engineered corn plants. A portion of or all of the seed can be the source of the degrading enzyme with other plant parts used for other purposes. The option is available to use a select tissue of the seed for commercial purpose, and other tissue used as the source of enzyme for the saccarification process. For example, the corn endosperm can be used as a source of starch, corn stover from the engineered plants as lignocellulosic biomass and embryo as the enzyme source. Further economic advantages are obtained in harvesting the seeds in a first operation and the stover in a second operation such that both operations are carried out concurrently by employing single-pass harvest operations.

The methods of the invention involve producing one or more cell wall polysaccharide-degrading enzymes in a crop plant by transforming the plant with at least one nucleotide construct comprising a nucleotide sequence encoding a cell wall polysaccharide-degrading enzyme operably linked to a promoter that drives expression in the crop plant, more preferably in the crop plant seed or a portion thereof, such that the production of the commercially desired plant material is not forfeited by the production of the enzymes.

The methods further involve obtaining from the transformed plant, tissue that expresses the cell wall polysaccharide-degrading enzyme or enzymes, contacting lignocellulosic biomass with this plant tissue, and exposing the combination to conditions that are favorable for the degradation of cell wall polysaccharides into fermentable sugars. The fermentable sugars can then be utilized for the production of ethanol or other desired molecules using fermentation procedures that are known in the art.

The inventors have devised an integrated method for the economic saccharification of lignocellulosic biomass and its conversion into ethanol. It is, therefore, an object of the present invention to provide cost-effective methods for converting polysaccharides in lignocellulosic biomass into fermentable sugars. It is also an object of the present invention to genetically engineer plants to produce cell wall degrading enzymes at commercially high levels and use such enzymes in saccharification of polysaccharides. A still further object is to obtain both the source of polysaccharides and source of enzymes from one crop. Another object of the invention is to integrate efficient harvest methods such as single pass harvest with the genetic engineering of corn plants to cost effectively produce ethanol from corn stover. A further object of the invention is to produce commercially acceptable levels of polysaccharide-degrading enzymes in corn plants. Yet another object of the invention is to target the expression of polysaccharide-degrading enzymes to corn seeds, preferably to the germ portion of the seed. The objectives of this invention will become apparent in the description below. All references cited are incorporated herein by reference.

### BRIEF DESCRIPTION OF THE DRAWINGS

FIG. 1 is a schematic diagram which shows an embodiment of the invention which comprises an integrated process for the production of ethanol from corn stover.

FIG. 2 shows β-D-glucosidase nucleotide sequences useful in the invention. (SEQ ID NO: 1)

FIG. 3 shows E1 cellulose nucleotide sequences useful in the invention. (SEQ ID NO: 2)

FIG. 4 shows CBH1 nucleotide sequences useful in the invention. (SEQ ID NO: 3)

### DETAILED DESCRIPTION OF THE INVENTION

The present invention is drawn to cost-effective methods for producing ethanol from plant material, particularly lignocellulosic biomass. By “lignocellulosic biomass” is intended biomass that is comprised predominantly of plant cell walls and the components therein including, but not limited to, cellulose, hemicellulose, pectin, and lignin. Current methods for the production of ethanol, which utilize starch derived from corn grain, are not cost effective. The methods of the invention involve the use of lignocellulosic biomass that is currently under utilized for the production of ethanol. Such lignocellulosic biomass includes, for example, crop plant residues or other undesired plant material that may be left behind in the field after harvest or separated from the desired plant material. By “desired plant material” is intended the plant product that is the primary reason for commercially growing the plant. Such desired plant material can be any plant or plant part or plant product that has commercial value. Corn is grown for human and animal consumption, as well as to produce products such as industrial oils, fertilizer and many other uses. Soybeans and wheat are used in food products. There are multitudes of purposes for which these plant materials can be utilized. The desired plant material also includes protein produced by a transgenic polynucleotide. In short, the desired plant material refers to any product from the plant that is useful. The invention allows for profitable use of what would otherwise could be low value or waste material after the desired plant is obtained. By a “crop plant” is intended any plant that is cultivated for the purpose of producing plant material that is sought after by man for either oral consumption, or for utilization in an industrial, pharmaceutical, or commercial process. The plant seed used may be that of the original plant transformed with the enzyme, or can be a descendant obtained by crossing with the same plant or another plants, as described in the methods below.

While such lignocellulosic biomass contains vast amounts of polysaccharides, these polysaccharides are not readily fermentable into ethanol. These polysaccharides are constituents of plant cell walls and include, but are not limited to, cellulose, hemicellulose, and pectin. The present invention provides cost-effective methods that involve converting at least a portion of these polysaccharides, particularly the portion comprising cellulose, into a form that can be readily fermented into ethanol by the microorganisms that are presently used for ethanol production, namely yeasts and bacteria. The invention integrates the economical production of the enzymes required for the conversion of the polysaccharides in lignocellulosic biomass to ethanol with the production of the desired plant material and the simultaneous recovery of the desired material, the lignocellulosic raw material and the polysaccharide-degrading enzymes in a single harvest operation.

The methods of the invention involve the conversion of plant cell wall polysaccharides to fermentable sugars that can then be used in the production of ethanol or other desired molecules via fermentation methods known in the art. The use of the term “fermentable sugars” includes, but is not limited to, monosaccharides and disaccharides and also encompasses sugar derivatives such as, for example, sugar alcohols, sugar acids, amino sugars, and the like. The fermentable sugars of the invention encompass any sugar or sugar derivative that is capable of being fermented into ethanol via fermentation methods known in the art.

To convert the cell wall polysaccharides to fermentable sugars, the methods of the invention involve producing in plant tissues one or more enzymes that are capable of degrading plant cell wall polysaccharides. Preferably, such enzymes are produced at high levels. Such enzymes and the sequences encoding them are known in the art.

Current sources of cell wall polysaccharide-degrading enzymes are fungal and microbial cultures. Producing high levels of cell wall polysaccharide-degrading enzymes in plants, particularly in grain crops, is less expensive and thus lowers the total cost of producing ethanol from lignocellulosic biomass (Z. Nikolov and D. Hammes. 2002. “Production of Recombinant Proteins from Transgenic Crops” in Plants as Factories for Protein Production., E. E. Hood and J. A. Howard, Eds., Kluwer Academic Publishers, Dordrecht, the Netherlands pp. 159-174). Expression of enzymes in plants has several advantages. Plants are more economical to grow and can be far more readily produced in large quantities than microbes. In addition, plant material is easy to store and transport.

The methods can involve, one, two, three, four, five, or more of such enzymes. The enzymes are preferably produced in plant seeds, or in a particular portion thereof, such as, for example, in the embryo, endosperm, seed coat, bran or hull.

Nature has developed effective cellulose hydrolytic machinery, mostly microbial in origin, for recycling carbon from plant biomass in the environment. Without it, the global carbon cycle would not function. To date, many cellulase genes have been cloned and sequenced from a wide variety of bacteria, fungi and plants (Tomme et al. (1995) Microbial Physiology 37:1-81). Cellulases are a subset of the glycosyl hydrolase family of enzymes that have been grouped into at least 13 families based on protein sequence similarity, enzyme reaction mechanism, and protein fold motif. The three-dimensional structures of cellulases from several different families have also been determined.

One reason that cellulose utilization has not yet been commercially realized is due to the high cost of the large quantities of cellulase enzymes required for its complete hydrolysis. Approximately 1.3 million tons/yr of cellulase would be required to convert the 48 million tons of stover-derived cellulose to glucose. While the development of superior enzymes for processing of plant polymers is important, superior enzymes are of little value unless the means to produce them economically on a large scale are also available. The methods of the instant invention provide for the cost-effective production of cellulases and other polysaccharide-degrading enzymes in plants, particularly transgenic maize.

While several recombinant systems are available for protein production, plants provide some advantages that the other systems lack. Although foreign proteins have been produced in animal cell culture and transgenic animal expression systems, these expression systems are very expensive and time intensive, making them highly impractical for industrial enzyme production. Bacterial and fungal expression systems are relatively simple systems, but require a large initial investment for fermentation equipment. Furthermore, the scale-up of fermentation systems for the large volumes of enzyme required for biomass conversion is also impractical. Capital and operating costs of such a fermentative approach to producing cellulases are likely to be impractical due to the huge scale that will be required.

On the other hand, crop-based production systems offer an attractive cost-effective alternative for industrial enzyme production at the scale required for biomass conversion. Transgenic plants require the lowest capital investment (mainly for dedicated harvesting equipment and storage) of all production systems. Several plant systems have been tested for industrial enzyme production including tobacco, oilseeds, barley and maize (Hood and Woodard, (2002) Industrial Proteins Produced from Plants, In: Plants as Factories for Protein Production, EE Hood and JA Howard, eds., supra. Plant systems have also been used to express cellulases. Expression of cellulase has been reported in Arabidopsis and tobacco tissue culture cells (Ziegler et al. (2000) Molecular Breeding 6:37-46). Additionally, some preliminary work has been reported for potato (Dai et al. (2000) Molecular Breeding 6:277-285). Recent studies with tobacco and alfalfa have shown that cell wall polysaccharide-degrading enzymes can be expressed in these plants (Ziegelhoffer et. al. (1999) Molecular Breeding 5:309-318).

Plant seeds offer an excellent alternative to fermentation for the high-level production of industrial enzymes. Plant seeds are natural low water content storage organs that serve to stabilize packaged proteins (Kusnadi et al. (1998) Biotechnol. Prog. 14:149-155). Proteins are stable for months to years in the grain, and can be stored on site until formulation is required. Seeds of a commodity crop are particularly attractive for the cost-effective, large scale production of industrial enzymes. Of the commodity crops, Zea mays L. or corn is an excellent choice for several reasons. Corn is the largest crop in North America and is widely used as a feedstock for corn-based industries. In addition, methods for the high-level expression of recombinant proteins in corn seeds are known in the art (Hood and Woodard, 2002. supra).

The advantages of low cost and ability to scale up are further enhanced in applications where highly purified products are not required, as is the case for biomass conversion. The cost of protein production in the grain of commodity crops is very low because of the high expression levels attainable and the limited capital necessary for production. Also, as an example of the rapid scale-up potential, in one to one-and-a-half years (three growing cycles) a gram of extractable protein can be expanded to 8000 kg of extractable protein, assuming a conservative 200-fold increase per generation. There are essentially no new technical issues associated with this mode of scale-up. Established practices for harvesting, transporting, storage, and processing are already in place and can easily be modified to include a value-added co-product, such as enzymes.

The methods of the invention involve transforming a plant with at least one nucleotide construct comprising at least one nucleotide sequence encoding an enzyme that is capable of degrading plant cell wall polysaccharides. The nucleotide sequence is operably linked to a promoter that drives expression in a plant. Preferably, the promoter will preferentially direct expression to a particular plant tissue. More preferably, the promoter will provide high-level expression in a particular plant tissue. Most preferably, the promoter will provide high-level expression in a seed, or in a particular part of the seed, such as, for example, the embryo (sometimes referred to as the “germ”), endosperm, seed coat, bran or hull. By “high-level expression” is intended that an enzyme of the invention is present in the plant tissue at a level of at least about 0.1% dry weight.

In one embodiment of the invention, the methods involve two or more cell wall polysaccharide-degrading enzymes. By “cell wall polysaccharide-degrading enzyme” is intended any enzyme that can be utilized to promote the degradation of the plant cell wall polysaccharides into fermentable sugars. While the methods of the invention encompass the production of one or more cell wall polysaccharide-degrading enzymes in a single plant, two or more enzymes can be produced in separate plants. For example, a first plant can be transformed with a first nucleotide construct comprising a first promoter operably linked to a first nucleotide sequence encoding a first polysaccharide-degrading enzyme. A second plant can also be transformed with a second nucleotide construct comprising a second promoter operably linked to a second nucleotide sequence encoding a second cell wall polysaccharide-degrading enzyme. The first and second enzymes can then be employed to degrade cell wall polysaccharides either in combination or sequentially.

Alternatively, both enzymes can be produced in a single plant. This can be accomplished by any means known in the art for breeding plants such as, for example, cross pollination of the first and second plants that are described above and selection for plants from subsequent generations which express both the first and second enzymes. The plant breeding methods used herein are well known to one skilled in the art. For a discussion of plant breeding techniques, see Poehlman (1987) Breeding Field Crops. AVI Publication Co., Westport Conn. Many crop plants useful in this method are bred through techniques that take advantage of the plant's method of pollination. A plant is self-pollinating if pollen from one flower is transferred to the same or another flower of the same plant. A plant is cross-pollinated if the pollen comes from a flower on a different plant. For example, in Brassica, the plant is normally self sterile and can only be cross-pollinated unless, through discovery of a mutant or through genetic intervention, self compatibility is obtained. In self-pollinating species, such as rice, oats, wheat, barley, peas, beans, soybeans, tobacco and cotton, the male and female plants are anatomically juxtaposed. During natural pollination, the male reproductive organs of a given flower pollinate the female reproductive organs of the same flower. Maize plants (Zea mays L.) can be bred by both self-pollination and cross-pollination techniques. Maize has male flowers, located on the tassel, and female flowers, located on the ear, on the same plant. It can self or cross pollinate.

Pollination can be by any means, including but not limited to hand, wind or insect pollination, or mechanical contact between the male fertile and male sterile plant. For production of hybrid seeds on a commercial scale in most plant species pollination by wind or by insects is preferred. Stricter control of the pollination process can be achieved by using a variety of methods of make one plant pool male sterile, and the other the male fertile pollen donor. This can be accomplished by hand detassling, cytoplasmic male sterility, or control of male sterility through a variety of methods well known to the skilled breeder. Examples of more sophisticated male sterility systems include those described at Brar et al., U.S. Pat. Nos. 4,654,465 and 4,727,219 and Albertsen et al. U.S. Pat. Nos. 5,859,341 and 6,013,859.

Backcrossing methods may be used to introduce the gene into the plants. This technique has been used for decades to introduce traits into a plant. An example of a description of this and other plant breeding methodologies that are well known can be found in references such as “Plant Breeding Methodology” edit. Neal Jensen, John Wiley & Sons, Inc. (1988). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred gene from the nonrecurrent parent.

A single plant can also be transformed with both the first and second nucleotide constructs described above or with a single nucleotide construct comprising the first promoter operably linked to the first nucleotide sequence and the second promoter operably linked to the second nucleotide sequence. Furthermore, it is recognized that both the first and second promoters can be the same or different depending on whether or not it is desired to express the first and second enzymes at the same level, time, and/or tissue in a plant or in separate plants.

Furthermore, as noted, the plant can be also transformed using such methods with another nucleotide sequence which creates a desired plant product. Such product can the plant with increased value, where the expression provides insect resistance, disease resistance, herbicide resistance, increased yield, increased tolerance to environmental stress, increased or decreased starch, oil or protein content, for example. The protein expressed in the plant can also be the desired plant product itself. By way of example, but not limitation such products can include production of proteases in plants (See U.S. Pat. No. 6,087,558); production of aprotinin in plants (U.S. Pat. No. 5,824,870; production of avidin in plant (U.S. Pat. No. 5,767,379); production of viral vaccines in plants (U.S. Pat. No. 6,136,320); production of transmissible gastroenteritis and hepatitis vaccines in plants (U.S. Pat. Nos. 5,914,123 and 6,034,298).

The enzymes of the invention encompass enzymes that can be employed to degrade plant cell wall polysaccharides into fermentable sugars. Such enzymes are known in the art and include, but are not limited to, enzymes that can catalyze the degradation of cellulose, hemicellulose, and/or pectin. In particular, the methods of the invention are drawn to cellulose-degrading enzymes. By “cellulose-degrading enzyme” is intended any enzyme that can be utilized to promote the degradation of cellulose into fermentable sugars including, but not limited to, cellulases and glucosidases. For the degradation of cellulose, for example, two general types of cellulase enzymes can be employed. Cellulase enzymes which cleave the cellulose chain internally are referred to as endo-β-1,4-glucanases (E.C. 3.2.1.4) and serve to provide new reducing and non-reducing chain termini on which exo-β-1,4-glucanases (cellobiohydrolase, CBH; E.C. 3.2.1.91) can operate (Tomme et al. (1995) Microbial Physiology 37:1-81). Two types of exoglucanase have been described that differ in their approach to the cellulose chain. One type attacks the non-reducing end and the other attacks the reducing end. The product of the exoglucanase reaction is typically cellobiose, so a third activity, β-D-glucosidase (E.C. 3.2.1.21), is required to cleave cellobiose to glucose. The exoglucanase can also yield longer glucose chains (up to 6 glucose units) that will require a β-D-glucosidase activity to reduce their size. Relative to the other enzymes activities needed for degradation of cellulose into fermentable sugars, only a minor amount of the β-D-glucosidase activity is required. Therefore, while the methods of the invention encompass the production of such a glucosidase in a plant, the necessary glucosidase activity could be supplied by a downstream fermentative organism or from β-D-glucosidase enzyme that is added during saccharification and/or fermentation.

Nucleotide sequences encoding endo-β-1,4-glucanases, exo-β-1,4-glucanases, and β-D-glucosidases are known in the art. Nucleotide sequences encoding endo-β-1,4-glucanases include, but are not limited to, the nucleotide sequence having Accession No. U33212. Nucleotide sequences encoding exo-β-1,4-glucanases include, but are not limited to, the nucleotide sequence having Accession No. X69976. Nucleotide sequences encoding β-D-glucosidases include, but are not limited to, the nucleotide sequence having Accession No. U13672. See http://us.expasy.org/cgi-bin/lists?glycosid.txt.

In addition to cellulose-degrading enzymes, enzymes that degrade hemicellulose and pectin can also be employed in the methods of the invention. While it is recognized that the soluble sugars can be liberated from the hemicellulose portion of lignocellulosic biomass by incubation in dilute acid at high temperatures, enzymes can be also be employed in the methods of the instant invention to convert hemicellulose into fermentable sugars. Such enzymes that can be used to the convert the polysaccharides of the hemicellulose portion into fermentable sugars are known in the art and include, but are not limited to, endo-β-1,4-xylanases, endo-β-1,4-mannanases, endo-β-1,4-galactanases, endoxylanases, α-glucuronidases, α-arabinofuranosidases, and α-arabinosidases. Nucleotide sequences encoding such enzymes are also known in the art. See http://us.expasy.org/cgi-bin/lists?glycosid.txt. Furthermore, additional fermentable sugars can be liberated from the pectin portion via the use of enzymes such as, for example, pectinases. Nucleotide sequences encoding such enzymes are also known in the art. See, Fry, S. C. 1985. Primary cell wall metabolism. Oxford Surveys of Plant Molecular and Cell Biology, ed. B. J. Miflin. 2:1-42. Oxford: Clarendon.

Following the degradation or saccharification of cell wall polysaccharides, the fermentable sugars that result therefrom can be converted into ethanol via fermentation methods employing microorganisms, particularly yeasts and/or bacteria. Such microorganisms and methods of their use in ethanol production are known in the art. See, Sheehan 2001. “The road to Bioethanol: A strategic Perspective of the US Department of Energy's National Ethanol Program” In: Glucosyl Hydrolases For Biomass Conversion. ACS Symposium Series 769. American Chemical Society, Washington, D.C., herein incorporated by reference. Existing ethanol production methods that utilize corn grain as the biomass typically involve the use of yeast, particularly strains of Saccharomyces cerevisiae. Such strains can be utilized in the methods of the invention. While such strains may be preferred for the production of ethanol from glucose that is derived from the degradation of cellulose and/or starch, the methods of the present invention do not depend on the use of a particular microorganism, or of a strain thereof, or of any particular combination of said microorganisms and said strains.

Furthermore, it is recognized that the strains of Saccharomyces cerevisiae that are typically utilized in fermentative ethanol production from corn starch might not be able to utilize galacturonic acid and pentose sugars such as, for example, xylose and arabinose. However, strains of microorganisms are known in the art that are capable of fermenting these molecules into ethanol. For example, recombinant Saccharomyces strains have been produced that are capable of simultaneously fermenting glucose and xylose to ethanol. See, U.S. Pat. No. 5,789,210, herein incorporated by reference. Similarly, a recombinant Zymomonas mobilis strain has been produced that is capable of simultaneously fermenting glucose, xylose and arabinose to produce ethanol. See, U.S. No. 5,843,760; herein incorporated by reference. See, also U.S. Pat. Nos. 4,731,329, 4,812,410, 4,816,399, and 4,876,196, all of which are herein incorporated by reference. These patents disclose the use of Z. mobilis for the production of industrial ethanol from glucose-based feedstocks. Finally, a recombinant Escherichia coli strain has been disclosed that is able to convert pure galacturonic acid to ethanol with minimal acetate production. See, Doran et al. ((2000) Appl. Biochem. Biotechnol. 84-86:141-152); herein incorporated by reference.

The methods of the invention involve obtaining plant tissue that expresses at least one of the cell wall-polysaccharide-degrading enzymes of the invention and lignocellulosic biomass. Preferably, the plant tissue is a seed or part thereof. More preferably the plant tissue is a grain seed or part thereof. Most preferably, the plant tissue is a corn kernel or part thereof, such as, for example, an embryo that is also referred to as the germ. The lignocellulosic biomass can originate from the same plants as the plant tissue or from different plants. Preferably, the lignocellulosic biomass comprises plant residues. More preferably, the lignocellulosic biomass comprises crop residues left in the field after the harvest of corn grain, which are also known as corn stover. Most preferably, the lignocellulosic biomass comprises corn stover that is from the same plants as the cell wall polysaccharide-degrading enzymes for increased cost efficiency.

The lignocellulosic biomass is contacted with the plant tissue and exposed to conditions favorable for the degradation of the polysaccharides in the lignocellulosic biomass. Prior to contacting the lignocellulosic biomass with the plant tissue, the plant tissue, the lignocellulosic biomass, or both, can be pretreated or processed in any manner known in the art that would enhance the degradation of the polysaccharides. For example, the lignocellulosic biomass can be processed by being chopped, sliced, minced, ground, pulverized, crushed, mashed or soaked. The plant tissue, such as the seed, containing the enzymes can be treated with dry or wet-milling processes. Such processing can also include incubating the plant tissue and/or lignocellulosic biomass in a solution, particularly an aqueous solution. If desired, the solution can be agitated, mixed, or stirred. The solution can comprise any components known in the art that would favor extraction of an active enzyme from the plant tissue and/or enhance the degradation of cell wall polysaccharides in the lignocellulosic biomass. Such components include, but are not limited to, salts, acids, bases, chelators, detergents, antioxidants, polyvinylpyrrolidone (PVP), polyvinylpolypyrrolidone (PVPP), and SO₂. Furthermore, specific environmental conditions, such as, for example, temperature, pressure, pH, O₂ concentration, CO₂ concentration, and ionic strength, can be controlled during any processing and/or subsequent steps to enhance polysaccharide degradation and/or ethanol production.

In certain embodiments of the invention, it may be desired to process the plant tissue so as to produce an extract comprising the polysaccharide-degrading enzyme and then contacting the lignocellulosic biomass with the extract. The processing of the plant tissue to prepare such an extract can be accomplished as described supra, or by any method known in the art for the extraction of an enzyme from plant tissue. In other embodiments of the invention, the plant tissue and the lignocellulosic biomass may be combined and then processed as described supra. See, e.g., Henry & Orit (1989) anal. Biochem. 114:92-96.

In yet another embodiment of the invention, prior to contacting the lignocellulosic biomass with the plant tissue or extract thereof, the lignocellulosic biomass can be prepared by pretreating the lignocellulosic biomass by methods known in the art (Nguyen et al. 1996. NREL/DOE Ethanol Pilot Plant: Current Status and Capabilities. Bioresource Technology 58:189-196). In the pretreatment step, the hemicellulosic fraction of the feedstock is hydrolyzed to soluble sugars. This step also increases the cellulase's ability to convert the major fraction of the feedstock (cellulose) to soluble glucose. The pretreatment step mixes the feedstock with sulfuric acid and water (approximately 1% acid in the final solution), then raises the slurry (20-25% solids) to reaction temperature (160-200° C.) with steam. The mixture is held at the reaction temperature for a predetermined time (2-20 min) then flashed into a tank maintained at near atmospheric pressure. Because of the sudden pressure drop, a fraction of the steam condensate and volatile compounds formed during the heating is evaporated and removed as flash tank overhead, which is condensed and sent to waste treatment. Lime is added to the remaining slurry to adjust the pH to 4.5.

While the cell wall polysaccharides are degraded prior to utilization of the fermentable sugars by microorganisms, the methods are not limited to a saccharification step which precedes the fermentation step. In certain embodiments of the invention, a single combined saccharification/fermentation step can be employed in the methods of the invention. In other embodiments, saccharification is initiated before fermentation and can be fully or partially complete prior to the initiation of the fermentation.

In a preferred embodiment of the invention, a corn plant is transformed with a nucleotide construct comprising at least one nucleotide sequence encoding a polysaccharide-degrading enzyme selected from the group consisting of an endo-β-1,4-glucanase, an exo-β-1,4-glucanase, and a β-D-glucosidase. The nucleotide sequence is operably linked to a promoter that drives expression in a plant cell, particularly in a seed cell, more particularly in the embryo cells (also referred to as germ cells), or the seed coat cells or hulls of the seed. Preferably, the desired polysaccharide-degrading enzyme or enzymes are expressed in the seed or embryo at a high-level, corresponding to at least about 0.1% of the dry weight of the seed. Corn plants that are bred or genetically engineered to comprise stably incorporated in their genomes the nucleotide constructs, are grown and kernels are produced therefrom that comprise the polysaccharide-degrading enzyme or enzymes. Preferably, such corn plants will serve as a source of both kernels comprising polysaccharide-degrading enzymes and corn stover that can be utilized as lignocellulosic biomass. In a preferred embodiment of the invention, both corn seeds and corn stover are harvested by a single harvesting operation. Such a procedure allows for the cost-effective recovery of both the seeds and the stover in one pass through the field. Using this procedure the seeds are collected in a first container and the corn stover in a second container and the collection of both the seeds and the stover is carried out concurrently in a single step. Single pass harvest integrates the collection of the lignocellulosic biomass with normal crop harvest operations. With this procedure the crop residues are collected without incurring a significant additional cost to the cost of harvesting the corn crop and without causing any additional soil compaction to cultivated fields from the passage of farm machinery, with decreased time and overall costs. Such a process has been demonstrated by Quick, G. R. (Oct. 29, 2001) Corn Stover Harvesting Filed Deomonstration and Biomass Harvesting Colloquium, Harlan, Iowa (record and minutes of program). In this particular process an IH 1460 with a John Deere 653A row crop head was coupled to a Hesston Stakhand wagon. The machines were modified by the Iowa State Agriculture Engineering department so that two crop streams were provided. Grain was taken up into the combine bin, and whole stover with cobs collected out the back of the machine and conveyed into the Stakhand wagon. This is just one example of the type of machine that can be used in such single pass harvesting.

Following harvest, the kernels can be milled either by the wet or dry milling methods that are known in the art. When the germ is to be separated from the seed, to be practical in this process, the germ should be capable of being separated in a commercial milling process, that is a process which does not require hand separation, but can be carried out in a commercial operation. Corn seed, for example, is readily separated from the germ or embryo, where soybean embryos are of a size that the only option for separation is by hand. In instances where the only means of separation of germ is by hand, the process would not provide the cost effective advantages as provided here.

There are two major milling processes for corn. Dry milling of corn separates the germ from the endosperm. The endosperm is recovered in the form of coarse grit and corn flakes, or it may be passed through fine rollers and reduced to corn flower.

The bulk of the corn starch produced in the United States is prepared by the wet-milling process. The first step in the wet-milling process is to steep the corn kernels in an aqueous solution. Steeping the kernels serves two main purposes. First it softens the kernels for subsequent milling, and second, it allows undesired soluble proteins, peptides, minerals and other components to be extracted from the kernels. After steeping, the kernels are separated from the steep water and then wet milled. The steep water is typically concentrated by evaporation to yield a solution referred to as a corn steep liquor. Corn steep liquor typically contains about 3.5 pounds dry solids per bushel of corn kernels with a nitrogen content between 45-48% (Blanchard (1992) Technology of Corn Wet Milling and Associated Processes, Elsevier, New York). Protein content in corn steep liquor has been estimated at about one pound per bushel of steeped corn which amounts to approximately 15-20% (w/w) of total corn kernel protein (Blanchard (1992) Technology of Corn Wet Milling and Associated Processes, Elsevier, New York).

While typical corn wet-milling processes employ a steeping that ranges from 12 to 48 hours, other wet-milling processes such as, for example, those known as the dry-grind process and the intermittent-milling-and-dynamic-steeping process involve an initial steeping of shorter duration and can additionally involve steeping at a higher temperature. Typically, the dry-grind and intermittent-milling-and-dynamic-steeping processes involve a steeping of whole kernels for about 12 hours or less at temperatures of about 60° C. The main objective of such a short initial steeping is to hydrate the embryo or germ. Breaking open the kernel after such a short initial steeping reduces the damage to the germ as compared to dry milling. The hydrated germ can then be recovered by methods typically utilized in the wet-milling process. The degerminated kernel fraction can then be subjected to a second steeping with additional grinding or milling to facilitate removal of soluble material from the kernel particles. See, Singh and Eckhoff (1996) Cereal Chem. 73:716-720 and Lopes-Filho et al. (1997) Cereal Chem. 74:633-638; herein incorporated by reference.

While the invention does not depend on the use of either dry or wet milling, it is recognized that either milling method can be used to separate the germ from the endosperm. By expressing the cell wall polysaccharide-degrading enzymes of the invention under the control of an embryo-preferred promoter, these enzymes can be preferentially produced in the corn germ. Thus, the isolated germ can be used as a source of enzymes for cell wall polysaccharide degradation, and the starch-laden endosperm can be utilized for other purposes. If desired, oil can also be extracted from the germ, using solvents such as, for example, hexane, before the germ is contacted with corn stover. Methods for extracting oil from corn germ are known in the art.

With wet-milling, the desired polysaccharide-degrading enzymes can be separated from the starch. As described above, a promoter that drives expression in an embryo, particularly a promoter that preferentially drives expression in the corn germ, can be operably linked to a nucleotide sequence encoding a polysaccharide-degrading enzyme of the invention. Because the germ is separated from the starch during wet milling, the germ, in the substantial absence of kernel starch, can be used as the enzyme source for degradation of cell wall polysaccharides in the corn stover. While the corn starch can be used for any purpose or in any process known in the art, the starch can also be used for the production of ethanol by methods known in the art. If desired, the starch can be used for ethanol production together with the corn stover. Thus, the starch can be recombined with the germ or combined with the stover or the stover-germ mixture. Starch-degrading enzymes are then utilized to degrade the starch into glucose for fermentation into ethanol.

Although the methods of the invention can be used for the saccharification of plant cell wall polysaccharides and the subsequent fermentation into ethanol, the invention does not depend on the production of ethanol. The invention encompasses any fermentative method known in the art that can utilize the fermentable sugars that are produced as disclosed herein. Such fermentative methods also include, but are not limited to those methods that can be used to produce lactic acid, malonic acid and succinic acid. Such organic acids can be used as precursors for the synthesis of a variety of chemical products that can be used as replacements for similar products that are currently produced by petroleum-based methods. See, United States Department of Energy Fact Sheets DOE99-IOFC17 (1999), DOE99-IOFC21 (1999), and DOE/GO-102001-1458 (2001); all of which are herein incorporated by reference.

If desired, the polysaccharide-degrading enzymes of the invention can be targeted to specific cellular compartments or organelles such as, for example, the cytosol, the vacuole, the nucleus, the endoplasmic reticulum, the mitochondria, the peroxisomes, and the plastids. Such targeting to specific compartments or organelles may increase the level of enzyme activity available to degrade cell wall polysaccharide in the methods of the invention. For example, if a desired polysaccharide-degrading enzyme of the invention is secreted from a plant cell when expressed therein, the nucleotide sequence encoding the amino acid sequence of this enzyme can be altered so as to prevent secretion. It is recognized that the localization of a nuclear-encoded protein within the cell is determined by the amino acid sequence of the protein and that the localization of an enzyme or protein can be altered by modifying the nucleotide sequence that encodes the protein in such a manner as to alter the amino acid sequence of the protein. Thus, the nucleotide sequences of the invention can be altered to redirect the cellular localization of the encoded proteins by any methods known in the art. Generally, such alterations involve modifying the nucleotide sequence encoding the protein in such a manner as to add or remove specific amino acids from the protein encoded thereby. Modifications include, but are not limited to, additions, deletions, and substitutions. For example, a cytosolic protein can be redirected to the plastid by operably linking a nucleotide sequence encoding a plastid transit peptide to the nucleotide sequence encoding a cytosolic protein. Such transit peptides are known in the art. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

The use of the term “nucleotide constructs” herein is not intended to limit the present invention to nucleotide constructs comprising DNA. Those of ordinary skill in the art will recognize that nucleotide constructs, particularly polynucleotides and oligonucleotides, comprised of ribonucleotides and combinations of ribonucleotides and deoxyribonucleotides may also be employed in the methods disclosed herein. Thus, the nucleotide constructs of the present invention encompass all nucleotide constructs that can be employed in the methods of the present invention for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs of the invention also encompass all forms of nucleotide constructs including, but not limited to, single-stranded forms, double-stranded forms, hairpins, stem-and-loop structures, and the like.

The nucleotide constructs of the invention encompass expression cassettes for expression in the plant of interest. The cassette will include 5′ and 3′ regulatory sequences operably linked to a nucleotide sequence encoding a polysaccharide-degrading enzyme of the invention. By “operably linked” is intended a functional linkage between a promoter and a second sequence, wherein the promoter sequence initiates and mediates transcription of the nucleotide sequence corresponding to the second sequence. Generally, operably linked means that the nucleotide sequences being linked are contiguous and, where necessary to join two protein coding regions, contiguous and in the same reading frame. The nucleotide construct can additionally contain at least one additional gene, such as for example, a selectable marker gene.

Such an expression cassette is provided with a plurality of restriction sites for insertion of the coding sequence for the polysaccharide-degrading enzyme of the invention to be under the transcriptional regulation of the regulatory regions.

The expression cassette will include in the 5′-3′ direction of transcription, a transcriptional and translational initiation region, a coding sequence for a polysaccharide-degrading enzyme of the invention, and a transcriptional and translational termination region functional in plants. The transcriptional initiation region, the promoter, can be native or analogous or foreign or heterologous to the plant host. Additionally, the promoter can be the natural sequence or alternatively a synthetic sequence. By “foreign” is intended that the transcriptional initiation region is not found in the native plant into which the transcriptional initiation region is introduced.

In addition to a promoter, the expression cassette can include one or more enhancers. By “enhancer” is intended a cis-acting sequence that increases the utilization of a promoter. Such enhancers can be native to a gene or from a heterologous gene. Further, it is recognized that some promoters can contain one or more native, enhancers or enhancer-like elements.

The termination region can be native with the transcriptional initiation region, can be native with the operably linked DNA sequence of interest, or can be derived from another source. Convenient termination regions are available from the Ti-plasmid of A. tumefaciens, such as the octopine synthase and nopaline synthase termination regions. In one embodiment of the invention the pin II terminator from the protease inhibitor II gene from potato (An et al., 1989. Functional analysis of the 3′ control region of the potato wound-inducible proteinase inhibitor II gene. Plant Cell 1:115-122) is used. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, the gene(s) may be optimized for increased expression in the transformed plant. That is, the genes can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, 5,436,391, and Murray et al. (1989) Nucleic Acids Res. 17:477-498, herein incorporated by reference.

Additional sequence modifications are known to enhance gene expression in a plant. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes can additionally contain 5′-leader sequences in the expression cassette construct. Such leader sequences can act to enhance translation. Translation leaders are known in the art and include but are not limited to: picornavirus leaders, for example, potyvirus leaders such as the TEV leader (Tobacco Etch Virus) (Allison et al (1986); MDMV leader (Maize Dwarf Mosaic Virus); Virology 154:9-20), untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Czech (Liss, New York), pp. 237-256); and maize chlorotic mottle virus leader (MCMV) (Lommel et al. (1991) Virology 81:382-385). Other methods known to enhance translation can also be utilized, for example, introns, and the like.

In preparing the nucleotide construct, the various DNA fragments can be manipulated, so as to provide for the DNA sequences in the proper orientation and, as appropriate, in the proper reading frame. Toward this end, adapters or linkers can be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

In the methods of the invention, a number of promoters that direct expression of a gene in a plant can be employed. Such promoters can be selected from constitutive, chemically-regulated, inducible, tissue-specific, and seed-preferred promoters. Constitutive promoters include, for example, the core CaMV 35S promoter (Odell et al. (1985) Nature 313:810-812); rice actin (McElroy et al. (1990) Plant Cell 2:163-171); ubiquitin (Christensen et al. (1989) Plant Mol Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730), and the like. Other constitutive promoters include, for example, U.S. Pat. Nos. 5,608,149; 5,608,144; 5,604,121; 5,569,597; 5,466,785; 5,399,680; 5,268,463; and 5,608,142.

Chemically-regulated promoters can be used to modulate the expression of a gene in a plant through the application of an exogenous chemical regulator. Chemically-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

The preferred promoters of the invention are seed-preferred promoters that are active during seed development. For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa zein, γ-zein, waxy, shrunken 1, shrunken 2, globulin 1, etc. Seed-preferred promoters of particular interest are those promoters that direct gene expression predominantly to specific tissues within the seed such as, for example, the endosperm-preferred promoter of γ-zein, the cryptic promoter from tobacco (Fobert et al. 1994. T-DNA tagging of a seed coat-specific cryptic promoter in tobacco. Plant J. 4: 567-577), the P-gene promoter from corn (Chopra et al. 1996. Alleles of the maize P gene with distinct tissue specificities encode Myb-homologous proteins with C-terminal replacements. Plant Cell 7:1149-1158, Erratum in Plant Cell.1997, 1:109), the globulin-1 promoter from corn (Blenger and Kriz. 1991. Molecular basis for Allelic Polymorphism of the maize Globulin-1 gene. Genetics 129: 863-972), and promoters that direct expression to the seed coat or hull of corn kernels, for example the pericarp-specific glutamine synthetase promoter (Muhitch et al., 2002. Isolation of a Promoter Sequence From the Glutamine Synthetase₁₋₂ Gene Capable of Conferring Tissue-Specific Gene Expression in Transgenic Maize. Plant Science 163:865-872). The Genbank accession number is: AF359511.

The methods of the invention involve transforming a plant cell with a nucleotide construct comprising a nucleotide sequence encoding a polysaccharide-degrading enzyme. The methods of the invention do not depend on a particular method for transforming plant cells with such a nucleotide construct, only that the production of the polysaccharide-degrading enzyme therein depends on the nucleotide construct. Methods for transforming plant cells with a nucleotide construct are known in the art including, but not limited to stable transformation methods, transient transformation methods, and viral methods.

By “stable transformation” is intended that the nucleotide construct introduced into a plant integrates into the genome of the plant and is capable of being inherited by progeny thereof. By “transient transformation” is intended that a nucleotide construct introduced into a plant does not integrate into the genome of the plant.

Methods for introducing expression vectors into plant tissue available to one skilled in the art are varied and will depend on the plant selected. Procedures for transforming a wide variety of plant species are well known and described throughout the literature. See, for example, Miki et al, supra; Klein et al. 1992. Bio/Technology 10:26; and Weisinger et al. 1988. Ann. Rev. Genet. 22: 421-477. For example, the DNA construct may be introduced into the genomic DNA of the plant cell using techniques such as microprojectile-mediated delivery (Klein et al. 1987. Nature 327: 70-73); electroporation (Fromm et al. 1985. Proc. Natl. Acad. Sci. 82: 5824); polyethylene glycol (PEG) precipitation (Paszkowski et al. 1984. Embo J. 3: 2717-272); direct gene transfer (WO 85/01856 and EP No. 0 275 069); in vitro protoplast transformation (U.S. Pat. No. 4,684,611) and microinjection of plant cell protoplasts or embryogenic callus (Crossway, 1985. Mol. Gen. Genetics 202:179-185). Co-cultivation of plant tissue with Agrobacterium tumefaciens is another option, where the DNA constructs are placed into a binary vector system (Ishida et al. 1996. “High Efficiency Transformation of Maize (Zea mays L.) Mediated by Agrobacterium tumefaciens”. Nature Biotechnology 14:745-750). The virulence functions of the Agrobacterium tumefaciens host will direct the insertion of the construct into the plant cell DNA when the cell is infected by the bacteria. See, for example Horsch et al. 1984. Science 233: 496-498, and Fraley et al. 1983. Proc. Natl. Acad. Sci. 80: 4803.

Corn transformation is described by Fromm et al, 1990. Bio/Technology 8:833 and Gordon-Kamm et al, supra. Agrobacterium is primarily used in dicots, but certain monocots such as maize can be transformed by Agrobacterium. U.S. Pat. No. 5,550,318. Rice transformation is described by Hiei et al. 1994. “Efficient Transformation of Rice (Oryza sativs L.) Mediated by Agrobacterium and Sequence Analysis of the Boundaries of the T-DNA” The Plant Journal 6(2): 271-282, Christou et al. 1992. Trends in Biotechnology 10:239 and Lee et al. 1991. Proc. Nat'l Acad. Sci. USA 88:6389. Wheat can be transformed by techniques similar to those used for transforming corn or rice. Sorghum transformation is described by Casas et al., 1997. Transgenic sorghum plants obtained after microprojectile bombardment of immature inflorescences. In vitro cellular and developmental biology, Plant. 33:92-100 and by Wan et al. 1994. Plant Physiology. 104:37. Soybean transformation is described in a number of publications, including U.S. Pat. No. 5,015,580.

In one preferred method, the Agrobacterium transformation methods of Ishida supra and also described in U.S. Pat. No. 5,591,616, are generally followed, with modifications that the inventors have found improve the number of transformants obtained. The Ishida method uses the A188 variety of maize that produces Type I callus in culture. In one preferred embodiment the High II maize line is used which initiates Type II embryogenic callus in culture. While Ishida recommends selection on phosphinothricin when using the bar or PAT gene for selection, another preferred embodiment provides for use of bialaphos instead.

The bacterial strain used in the Ishida protocol is LBA4404 with the 40 kb super binary plasmid containing three vir loci from the hypervirulent A281 strain. The plasmid has resistance to tetracycline. The cloning vector cointegrates with the super binary plasmid. Since the cloning vector has an E. coli specific replication origin, but not an Agrobacterium replication origin, it cannot survive in Agrobacterium without cointegrating with the super binary plasmid. Since the LBA4404 strain is not highly virulent, and has limited application without the super binary plasmid, the inventors have found in yet another embodiment that the EHA 101 strain is preferred. It is a disarmed helper strain derived from the hypervirulent A281 strain. The cointegrated super binary/cloning vector from the LBA4404 parent is isolated and electroporated into EHA 101, selecting for spectinomycin resistance. The plasmid is isolated to assure that the EHA101 contains the plasmid.

Further, the Ishida protocol as described provides for growing fresh culture of the Agrobacterium on plates, scraping the bacteria from the plates, and resuspending in the co-culture medium as stated in the '616 patent for incubation with the maize embryos. This medium includes 4.3 g MS salts, 0.5 mg nicotinic acid, 0.5 mg pyridoxine hydrochloride, 1.0 ml thiamine hydrochloride, casamino acids, 1.5 mg 2,4-D, 68.5 g sucrose and 36 g glucose, all at a pH of 5.8. In a further preferred method, the bacteria are grown overnight in a 1 ml culture, then a fresh 10 ml culture re-inoculated the next day when transformation is to occur. The bacteria grow into log phase, and are harvested at a density of no more than OD600=0.5 and is preferably between 0.2 and 0.5. The bacteria are then centrifuged to remove the media and resuspended in the co-culture medium. Since Hi II is used, medium preferred for Hi II is used. This medium is described in considerable detail by Armstrong, C. I. and Green C. E. 1985. Establishment and maintenance of friable, embryogenic maize callus and involvement of L-proline. Planta 154:207-214. The resuspension medium is the same as that described above. All further Hi II media are as described in Armstrong et al. The result is redifferentiation of the plant cells and regeneration into a plant. Redifferentiation is sometimes referred to as dedifferentiation, but the former term more accurately describes the process where the cell begins with a form and identity, is placed on a medium in which it loses that identity, and becomes “reprogrammed” to have a new identity. Thus the scutellum cells become embryogenic callus.

Transformed plant cells and tissues can be regenerated into plants by standard methods. See, for example, McCormick et al. (1986) Plant Cell Reports 5:81-84. These plants may then be grown, and either pollinated with the same transformed strain or different strains, and the resulting plants producing the desired polysaccharide-degrading enzyme of the invention. Two or more generations may be grown to ensure that production of the desired enzyme is stably maintained and inherited and then seeds harvested and tested to ensure they possess the desired enzyme.

In a preferred embodiment, the expression vector also contains a gene encoding a selection marker that is functionally linked to a promoter that controls transcription initiation. For a general description of plant expression vectors and reporter genes, see Gruber et al. 1993. “Vectors for Plant Transformation” in Methods of Plant Molecular Biology and Biotechnology. CRC Press. p 89-119. In a preferred embodiment, the selective gene is a glufosinate-resistance encoding DNA and in another preferred embodiment can be the phosphinothricin acetyl transferase (“PAT”) or maize optimized PAT gene under the control of the CaMV 35S promoter. The gene confers resistance to bialaphos (Gordon-Kamm. 1990. The Plant Cell 2: 603; Uchimiya et al. 1993. Bio/Technology 11: 835; and Anzai et al, 1989. Mol. Gen. Gen. 219: 492.

The methods of the invention find use with any plant species capable of producing a polysaccharide-degrading enzyme of the invention. Preferably, the plant species are crop plant species. More preferably, the plant species are selected from the grain and oilseed plants. Most preferably, the plant species is corn.

The following example is offered by way of illustration and not by way of limitation.

### EXAMPLE 1

### A Cost-Effective System for Sugar Production from Lignocellulosic Biomass

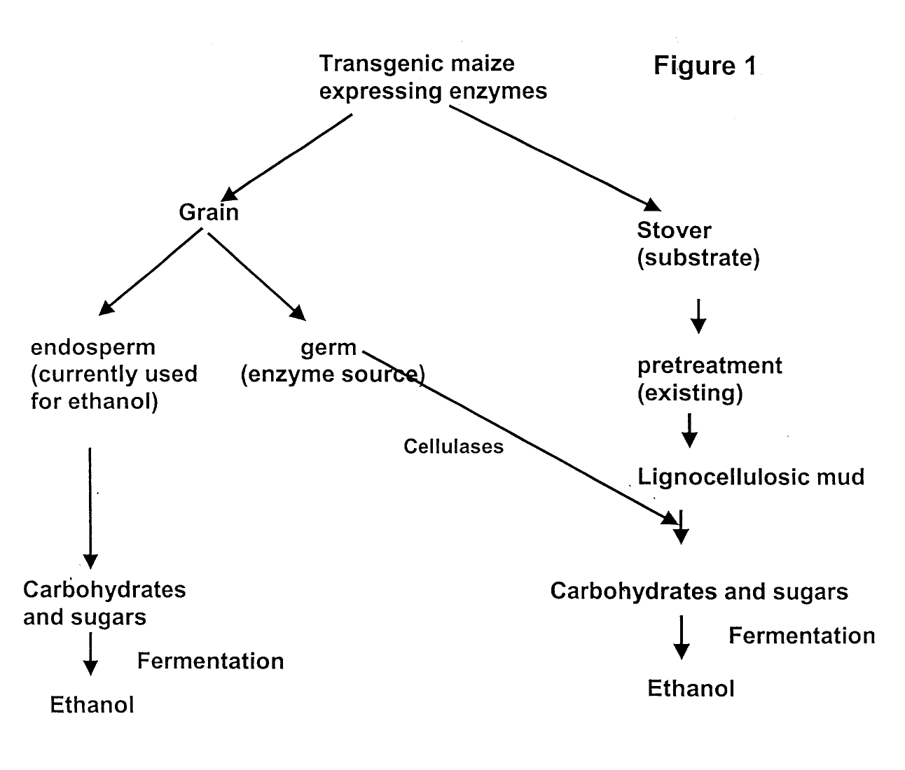

In an embodiment of the invention, maize plants are genetically engineered to produce large amounts (beginning at 0.1% of whole seed or embryo dry weight) of active bacterial or fungal cellulase enzymes in grain. Corn grain that expresses the desired cellulases is grown and harvested. The corn grain can be economically transported (low water content) and fractionated using either a wet or dry milling process to produce a cellulase-rich fraction that can be employed in conversion of a variety of lignocellulosic feedstocks. The paradigm illustrated in FIG. 1 is even more cost-effective if a single pass harvesting of stover—the lignocellullosic biomass feedstock—and grain—the enzyme source—can be implemented.

Corn plants are transformed with one or more nucleotide sequences encoding polysaccharide-degrading enzymes, particularly cellulose-degrading enzymes. Such nucleotide sequences are known in the art. For example, more than 400 cellulase genes/enzymes including endo- and exoglucanases have been described from fungi, bacteria and plants (Tomme et al. (1995) Advances in Microbial Physiology 37:1-81; Schulein (2000) Biochim. Biophys. Acta 1543:239-252). These genes/enzymes have been classified into 13 families based on sequence homology, protein domain structure and mechanism of catalysis. In one embodiment of the invention, cellulase enzymes having the following criteria are selected for expression in plants. These cellulase enzymes are thermostable to at least 45° C., have pH optima that are similar, exhibit synergistic activity on lignocellulosic substrates, and the genes encoding these enzymes have been cloned.

Using these criteria, the E1 β-1,4-endoglucanase from Acidothermus celluloliticus (Mohagheghi et al. (1986) Isolation and Characterization of Acidothermus cellulolyticus gen. Nov., sp. Nov., a new genus of thermophillic, acidophillic, cellulolytic bacteria. Int. J. Syst. Bacteriol. 36:435-443; Nieves et al. (1995) Appl. Biochem. Biotechnol. 51/52:211-223; U.S. Pat. No. 5,536,655), cellobiohydrolase I (CBHI) from Trichoderma reesii (Shoemaker et al. (1983). Molecular Cloning of Exo-Cellobiohydrolase I Derived From Trichoderma Reesei Strain L27. Bio/Technology 691-696) and the β-D-glucosidase from Candida wickerhamii (Skory and Freer (1995) Appl. Environ. Microbiol. 61:518-525; Freer (1993) J. Biol. Chem. 268:9337-9342) have been selected. This latter enzyme is a preferred glucosidase because it is resistant to feedback inhibition by glucose and cellobiose—an important consideration if one separates the process of saccharification from fermentation. If saccharification is performed separately from fermentation, the glucosidase should be selected which will not be feedback inhibited by their products. The first two enzymes—E1 and CBHI—have been shown to exhibit synergistic activity on lignocellulosic substrates that have been pretreated with dilute acid and steam (Baker et al. (1994) Appl. Biochem. Biotechnol. 45/46:245-256). E1 has optimal activity at 81° C. (Table 1) but is compatible at 45-50° C. with the CBHI enzyme which shows optimal and sustained activity at 50° C. Thermostable enzymes with high temperature optima are less likely to produce detrimental affects on plants during their growth and development at ambient temperatures. Some physical characteristics of the selected enzymes for this embodiment of the invention are presented in Table 1.

TABLE 1
Characteristics of Selected Cellulose-Degrading Enzymes

| | E1 cellulase | CBH I | β-glucosidase |
|---|---|---|---|
| Family | 5-3.2.1.4 | 7-3.2.1.91 | 1-3.2.1.21 |
| Calculated MW | 521 aa; 56,500 Da | 496 aa; 52,500 Da | 94,000 Da native |
| | | | 116,000 Da in yeast |
| Native source | Bacterial | Fungal | Fungal |
| (catalytic | 363 aa; 40,610 Da | | |
| domain) | | | |
| MW b SDS PAGE | 72,000 Da | 65,000 Da | 94,000 Da |
| (catalytic | 60,000 Da | | 72,000 non-glycosyl |
| domain) | | | |
| Glycosylated native | No | Yes, primarily | Yes, 30% |
| protein | | linker region | |
| pI | 5.2 (holo) | 4.51 | 3.89² |
| | 4.87 (cat domain) | | |
| pH optimum | 5-6 | 5 | 4.75 |
| Temperature | 81° C. | 45-50° C. | <45° C. |
| Bond cleaved | β-1,4-glycosidic | β-1,4-glycosidic | β1,4-glycosidic |
| Mechanism | Retained anomeric | Retained anomeric | Retained anomeric |
| | configuration¹ | configuration¹ | configuration¹ |
| Substrates | Cellulose fibrils; | Cellulose fibrils; | Cellobiose (and |
| | purified cellulose | purified cellulose | other water-soluble |
| | preparations (Solka- | preparations | cello-oligomers up |
| | floc, Sigmacell, | (Solka-floc, | to dp 6); other β-1,4- |
| | Avicel); para- | Sigmacell, Avicel) | glycosides (para- |
| | nitrophenyl-β-1,4- | | nitrophenyl-β-1,4- |
| | D-cellobiose | | D-glucose (pNPG); |
| | (pNPC); | | methylumbelliferyl- |
| | methylumbelliferyl- | | β-1,4-D-glucose |
| | β-1,4-D-cellobioside | | (MUG) |
| | (MUC) | | |
| Primary reaction | Decreased degree of | Cellobiose (and | Glucose |
| products | polymerization (dp), | other water-soluble | |
| | long-chain, water- | short chain cello- | |
| | insoluble cellulose | oligomers) | |

¹Schulein (2000) Biochim. Biophys. Acta 1543:239-252.
²Freer (1993) J. Biol. Chem. 268:9337-9342.

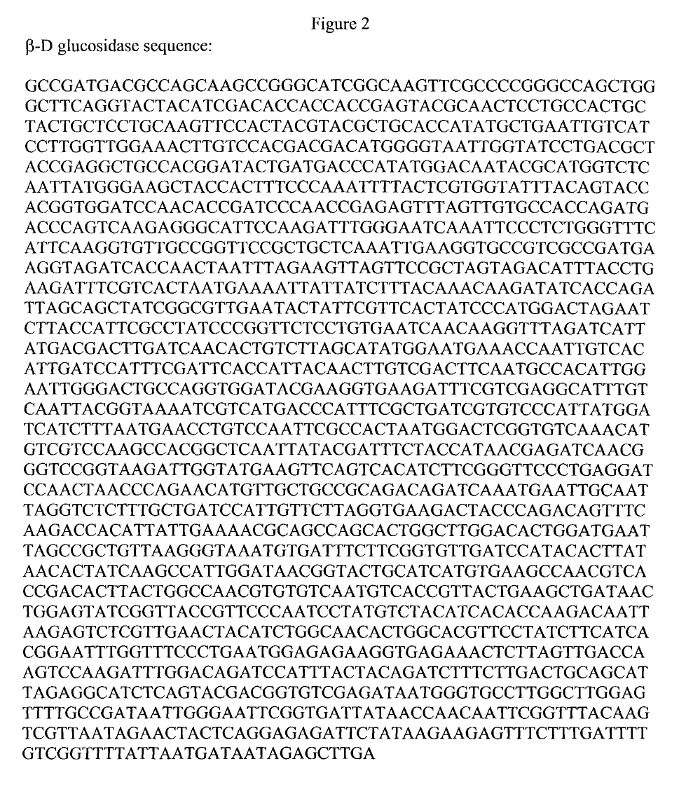

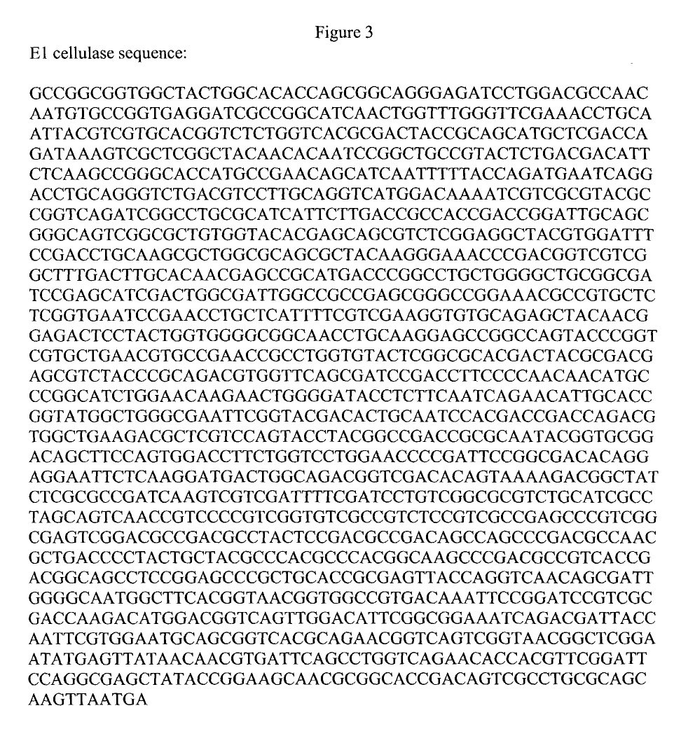

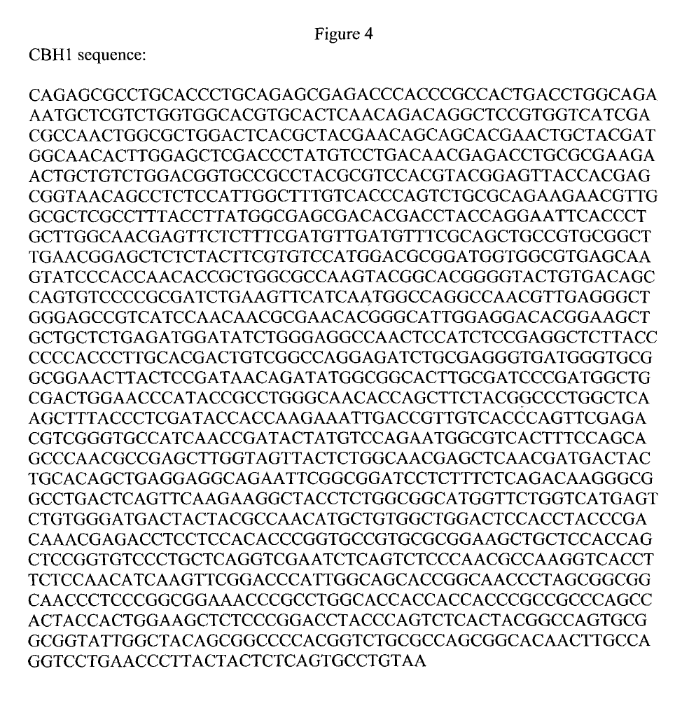

Expression cassettes are prepared for use in producing transformed plants. The expression cassettes comprise a selectable marker gene linked to a nucleotide construct comprising an embryo-preferred promoter operably linked to a nucleotide sequence encoding a cellulose-degrading enzyme. In a preferred embodiment of the invention, the globulin-1 promoter from maize is used, see Belanger and Kriz (1991), Molecular Basis for Allelic Polymorphism of the maize Globulin-1 gene. Genetics 129: 863-972). It has an accession number L22344 in the Genbank database. The constructs are indicated in Table 2. The nucleotide sequences encoding the cellulose-degrading enzymes are modified to direct expression to specific cellular organelles or to the outside of the cell or apoplast. Thus, the first step in the cloning process is to attach an organelle-targeting sequence to the target protein coding sequence using overlap extension PCR as described in Hood et al. (1997). Commercial production of avidin from transgenic maize: Characterization of transformant, production, processing, extraction and purification. Molecular Breeding 3: 291-306. The sequences for E1 cellulase is set forth in FIG. 2 (SEQ ID NO:1); CBH I at FIG. 3 (SEQ ID NO:2) and β-D-glucosidase at FIG. 4 (SEQ ID NO:3). Each of these sequences was combined with a BAASS signal on the 5′end, a 5′ end BAASS (Rogers, J. C. (1985). Two barley alpha-amylase gene families are regulated differently in aleurone cells. J. Biol Chem 260, 3731-3738) and a 3′end KDEL, (contains the binding site for a receptor in the endoplasmic reticulum. Munro, S. and Pelham, H. R. B. (1987)) or a vacuole signal seq on the 5′ end (Howerdar, B. C. et al., 1990 “In Vitro Processing of Aleurain, a Barley Vacuolar Thiol Protease” Plant Cell 11: 1091-1106). The first 40 amino acids were maize codon optimized for each construct (the signal sequence was included in that 40). The N terminal Methionine was also removed from each gene. The ATG of the signal sequence was used as the translational start site. In the CBH1 gene two codons (D346 and D386) were also changed to reduce possible message instability.

These sequences are then cloned into an expression cassette vector containing the desired promoter and the PinII terminator before transferring them into a final vector that also includes the selectable marker gene.

TABLE 2
Nucleotide Constructs for the Expression of
Cellulose-Degrading Enzymes in Transgenic Corn

| | | Target | Transcription |
|---|---|---|---|
| Coding Sequence | Promoter | Organelle | Terminator |
| E1 cellulase | Embryo-preferred | Cell wall | PinII |
| | Embryo-preferred | Vacuole | PinII |
| | Embryo-preferred | ER | PinII |
| CBH I | Embryo-preferred | Cell wall | PinII |
| | Embryo-preferred | Vacuole | PinII |
| | Embryo-preferred | ER | PinII |
| β-D-glucosidase | Embryo-preferred | Cell wall | PinII |
| | Embryo-preferred | Vacuole | PinII |
| | Embryo-preferred | ER | PinII |

The expression cassettes comprising the nucleotide constructs indicated in Table 2 are used to stably transform corn plants via Agrobacterium-mediated transformation. Initially, expression cassettes comprising the nucleotide constructs that are indicated in Table 2 are prepared and transferred to the super binary Agrobacterium tumefaciens strains described by Ishida et al. (1996). High Efficiency transformation of maize (Zea mays L.) mediated by Agrobacterium tumefaciens. Nature Biotechnology 14: 745-750). Fresh immature zygotic embryos were harvested from Hi II maize line at 1-2 mm in length. The fresh embryos were treated with 0.5 ml log phase Agrobacterium strains EHA 101. Bacteria were grown overnight in a rich medium with kanamycin and spectinomycin to an optical density of 0.5 at 600 nm, pelleted, then re-introduced in a fresh 10 ml culture. The bacteria were allowed to grow into log phase and were harvested at no more dense than OD 600=0.5. The bacterial culture is resuspended in a co-culture medium. For stable transformations, embryos not subjected to sonication were transferred to a bialaphos selective agent on embryogenic callus medium and transferred thereafter every two weeks to allow growth of the transformed type II callus. Plants were regenerated from the callus.

Transgenic plants from tissue culture (T₀ generation) are transferred to the greenhouse and potted in soil for seed production (T₁ seed). Approximately three months after transfer to soil, 50-150 seed are harvested from each plant, the amount of which is dependent on the vigor of the T₀ plant. These seed are utilized in biochemical assays to choose the highest expressing events and lines as well as being the seed source for subsequent multiplication of those lines in the field.

A variety of assays for endo-β-1,4-glucanase, cellobiohydrolase and β-D-glucosidase are known in the art which can be used to detect enzyme activity in extracts prepared from maize callus and seeds. See, Coughlan et al. ((1988) J. Biol. Chem. 263:16631-16636) and Freer ((1993) J. Biol. Chem. 268:9337-9342); both of which are herein incorporated by reference. In addition, western analysis and ELISAs can be used to assess protein integrity and expression levels. Individual T₁ seeds are screened by the assay of choice for expression of the target protein, in this case the cellulases or β-glucosidase. The individual plants expressing the highest levels of active enzyme are chosen for field studies, which include back-crosses (See “Plant Breeding Methodology” edit. Neal Jensen, John Wile & Sons, Inc. 1988), selection for increased expression and increased seed amounts. A Western analysis is a variation of the Southern analysis technique. With a Southern analysis, DNA is cut with restriction endonucleases and fractionated on an agarose gel to separate the DNA by molecular weight and then transferring to nylon membranes. It is then hybridized with the probe fragment which was radioactively labeled with ³²P and washed in an SDS solution. In the Western analysis, instead of isolating DNA, the protein of interest is extracted and placed on an acrylamide gel. The protein is then blotted onto a membrane and contacted with a labeling substance. See e.g., Hood et al., “Commercial Production of Avidin from Transgenic Maize; Characterization of Transformants, Production, Processing, Extraction and Purification” Molecular Breeding 3:291-306 (1997).

The ELISA or enzyme linked immunoassay has been known since 1971. In general, antigens solubilised in a buffer are coated on a plastic surface. When serum is added, antibodies can attach to the antigen on the solid phase. The presence or absence of these antibodies can be demonstrated when conjugated to an enzyme. Adding the appropriate substrate will detect the amount of bound conjugate which can be quantified. A common ELISA assay is one which uses biotinylated anti-(protein) polyclonal antibodies and an alkaline phosphatase conjugate. For example, an ELISA used for quantitative determination of laccase levels can be an antibody sandwich assay, which utilizes polyclonal rabbit antibodies obtained commercially. The antibody is conjugated to alkaline phosphatases for detection. In another example, an ELISA assay to detect trypsin or trypsinogen uses biotinylated anti-trypsin or anti-trypsinogen polyclonal antibodies and a streptavidin-alkaline phosphatase conjugate

An initial test of enzyme function is performed with lines of processed corn seed containing single enzymes. For saccharification of cellulose, seed tissue from these lines are mixed in the appropriate ratio to produce a high specific activity for degradation of crystalline cellulose. According to Baker et al. ((1995) “Synergism between purified bacterial and fungal cellulases”, in Enzymatic Degradation of Insoluble Carbohydrates. ACS Series 618, American Chemical Society, Washington, D. C, pp. 113-141.), maximum synergism for saccharification of cellulose is with a composite that is about 80% of the Trichoderma reesei CBHI (exo- -1,4-glucanas) and about 20% of the Acidothermus cellulolyticus endo- -1,4-glucanase. The addition of about 0.1% of the Candida wickerhamii-D-glucosidase facilitates the degradation of short glucose oligomers (dp=2-6) to yield glucose. Later, cross pollination of the selected lines is used to produce lines that express all three of the cellulase-degrading enzymes.

The corn crop is harvested using the one-pass harvest procedure. The kernels are collected in one bin and the corn stover in another bin at the same time. The kernels are later fractionated into the germ tissue containing the enzymes and the endosperm. The germ tissue or extracts thereof are used as the source of enzyme to convert cell wall cellulose into fermentable sugars. The endosperm is used as a source of starch. The corn stover is the source of lignocellulosic biomass. The fermentable sugars are used as a precursor for the synthesis of a variety of chemical compounds, for example ethanol.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A method for the cost-effective saccharification of polysaccharides in crop residues comprising the steps of:
a) transforming tissue of a crop plant which can produce seed with a nucleotide sequence encoding at least one polysaccharide-degrading enzyme such that the expression of the polysaccharide-degrading enzyme is preferentially expressed to the seed of the plant;
b) culturing and growing a plant from the plant tissue;
c) producing seed from the plant or descendants of the plant, such that the enzyme is expressed in the seed of the plant or descendant;
d) harvesting the plant or descendant to obtain the seeds of the plant or descendant and crop residues;
e) fractionating the seeds into separate tissues such that at least one of said tissues contains said enzyme;
f) contacting the crop residues with at least one of the enzymes contained in at least one of the separated tissues containing the enzyme; and
g) exposing the crop residues and at least one of the enzymes to conditions favorable for the degradation of polysaccharides in the crop residues so as to produce fermentable sugars.

2. The method of claim 1 comprising extracting the at least one enzyme prior to contacting the crop residues with the at least one enzyme.

3. The method of claim 1 comprising contacting the crop residues with at least one of the separated tissues containing the at least one enzyme.

4. The method of claim 1 comprising harvesting the seeds of the plant or descendant in a first operation and the crop residues in a second operation such that the first operation and the second operation are carried out concurrently in a single step.

5. The method of claim 1 comprising preferentially expressing the enzyme in the germ tissue of the seed, fractionating the germ tissue from the seed in a commercial milling process and contacting the crop residues with at least the germ tissue of the seed.

6. The method of claim 5 comprising preferentially expressing the enzyme in the germ tissue of the seed at levels of at least about 0.1% of total dry weight of the seed.

7. The method of claim 1 further comprising fermenting the fermentable sugars under conditions favorable for ethanol production so as to produce ethanol.

8. The method of claim 1 further comprising pretreating the crop residues prior to contacting the crop residues with the enzyme.

9. The method of claim 1 wherein the polysaccharide-degrading enzyme is selected from the group consisting essentially of: endo-β-1,4-glucanase, exo-β-1,4-glucanase, β-D-glucosidase, hemicellulases and pectinases.

10. The method of claim 1 wherein the polysaccharide-degrading enzyme is cellulase.

11. The method of claim 1 wherein the plant is bred with at least one other plant and descendants grown having seeds containing at least one of the enzymes.

12. The method of claim 11 wherein at least one of the plants is transformed with a nucleotide sequence expressing protein which produces desired plant material.

13. The method of claim 1 wherein the crop plant is corn.

14. A method for the cost-effective saccharification of polysaccharides in corn stover comprising the steps of:
a) transforming corn tissue with a nucleotide sequence encoding at least one polysaccharide-degrading enzyme such that the expression of the polysaccharide-degrading enzyme is preferentially expressed in the germ of the seed;
b) culturing a plant from the corn tissue;
c) growing the plant and producing seeds from the plant or descendants of the plant such that the enzyme is expressed in the seed of the plant or descendant;
d) harvesting the seeds of the plant or descendants in a first operation and the plant stover in a second operation such that the first operation and the second operation are carried out concurrently in a single step;
e) fractionating the germ from the seed;
f) contacting the corn stover with at least one member from the group consisting essentially of the germ, an extract of the germ and cells of the germ tissue; and
g) exposing the corn stover and the member to conditions favorable for the degradation of polysaccharides in the corn stover so as to produce fermentable sugars.

15. The method of claim 14 wherein the polysaccharide-degrading enzyme is produced in the germ at a level of at least about 0.1% of total dry weight of the seed.

16. The method of claim 14 further comprising fermenting the fermentable sugars under conditions favorable for ethanol production so as to produce ethanol.

17. The method of claim 14 further comprising pretreating the crop residues prior to contacting the crop residues with the member.

18. The method of claim 14 wherein the polysaccharide-degrading enzyme is selected from the group consisting essentially of: endo-β-1,4-glucanase, exo-β-1,4-glucanase, β-D-glucosidase, hemicellulases and pectinases.

19. The method of claim 14 wherein the polysaccharide-degrading enzyme is cellulase.

20. A method for the cost-effective saccharification of polysaccharides in crop residues comprising the steps of:
a) transforming tissue of a crop plant which can produce seed with a nucleotide sequence encoding at least one polysaccharide-degrading enzyme such that the expression of the polysaccharide-degrading enzyme is preferentially expressed to the seed of the plant;
b) culturing and growing a plant from the plant tissue;
c) producing seed from the plant or descendants of the plant, such that the enzyme is expressed in the seed of the plant or descendant;
d) harvesting the plant or descendant to obtain the seeds of the plant or descendant and crop residues;
e) contacting the crop residues with at least one of the enzymes contained in at the seed; and
f) exposing the crop residues and at least one of the enzyme to conditions favorable for the degradation of polysaccharides
